(19) United States
(12) Patent Application Publication
(10) Pub. No.: US 2008/0077075 A1
(43) Pub. Date: Mar. 27, 2008

(54) CONSTRUCTIVE DISPOSITION INTRODUCED IN INDEFLATOR EQUIPMENT APPLIED IN CORONARY ANGIOPLASTY INTERVENTION

(76) Inventors: Alexander Marra MOREIRA, Sao Paulo (BR); Melchiades da Cunha Neto, Goiania (BR)

Correspondence Address: STITES & HARBISON PLLC, 1199 NORTH FAIRFAX STREET, SUITE 900, ALEXANDRIA, VA, 22314, US

(21) Appl. No.: 11/535,144

(22) Filed: Sep. 26, 2006

(51) Int. Cl. A61N 1/30

(52) U.S. Cl. 604/19

(57) ABSTRACT

Constructive disposition introduced in indeflator equipment applied in coronary angioplasty intervention represented by an evolutionary solution translated by improvements of construction nature in the locking/unlocking system of the embolus, providing a better control over the injection or removal of liquids and/or fluids in a balloon catheter, being such locking/unlocking system (A) presents a simple construction concept, with bigger reliability and durability, making easy its assembling, with small quantity of pieces, minimizing the possibility of non-conform mechanical performance, and additionally providing easy handling by nurses and/or auxiliary people, being that the operation to inflate or to empty the balloon catheter with liquid or fluid is made possible by the embolus (B) movement over the syringe (D), through the application of a force to inflate (Fi) or to empty (Fe) in the hold element (C), through an embolus rotation, since to inflate the balloon the system must be locked, where to that the locking/unlocking system (1) now claimed presents a mechanism where the multiplicity of teeth of the inferior part (5b) of the crog rail component (5) acts directly over the thread region (B1) of the embolus (B), being the maintenance of this position made possible by a totally distended helicoidal spring (6) when in the locking position.

### BACKGROUND OF THE INVENTION

The present demand of patent of utility model of the mentioned title treats of an evolutionary solution to indeflator equipment, also known as insufflator equipment, widely used in surgical intervention, particularly in coronary angioplasty interventions, where it receives an improvement of constructive nature in the locking/unlocking system, thus allowing an efficient control of the liquids and/or fluids administration in balloon catheter.

The constructive concept now introduced also provides a greater reliability to the equipment, in durability terms, since it is characterized by easy assembly, involving small quantity of component parts, minimizing the possibility of non-conform mechanical performance.

Additionally the new locking/unlocking system of the embolus provides to the indeflator equipment an ergonomic feature, since it allows to the technician who handles it (for example, nurses and/or auxiliary people), a singular agility to use the instrument, avoiding physical stress conditions of his/her hands, and also allowing a better control of the liquids and/or fluids administration.

Thus it is conclusive that the improvement in the indeflator equipment now claimed has an inventive activity with industrial and commercial applications, accomplishing the requirements of patentability, specially as utility model, according to disposed on article 9^{th} of Law 9.279 (Lei de Patentes, Marcas e Direitos Conexos—Brazil), May 14, 1996.

### DESCRIPTION OF THE PRIOR ART

In order to provide veracity to the above described, an explanation of the state of the technique to indeflator equipment will be done, where it will be possible to a skilled person to recognize its limitative aspects, mainly about its construction, functionality and ergonomics, to afterwards discuss about the aggregate advantages with the introduction of the improvement in its embolus locking/unlocking system now claimed.

The indeflator equipment, also known in the surgical sector as insufflator equipment, has as a secondary function to provide the liquids and/or fluids administration in a balloon catheter.

At its turn, it has as main function, when used during surgical interventions, to inflate, to control the pressure and also to empty the balloon catheter, being this procedure widely used in angiography procedures, allowing the placement of endoprosthesis, also known as ‘stent’, for example.

At its turn, the insufflator equipment presents its constructive concept composed by the following main components:
Syringe;
Syringe embolus;
Manometer;
Control system to inflate or to empty the balloon catheter; and
Pipe to connect the catheter.

After recognizing the constructive concept of the insufflator equipment, it is important to make a value analysis related to its functional efficiency. After several observations and comments we can conclude that, besides the equipment accomplishes the requirements of “inflating, controlling the pressure and emptying the balloon catheter”, the same one presents deficient aspects of constructive and operational nature.

From the point of view of its construction it is conclusive that particularly its control system to inflate or to empty the balloon catheter presents a complex construction, which can lead to the following problems:
Hard assembly during manufacturing;
High index of functional non-conformity;
Limited durability;
Hard handling (equipment hold); and
Hard control of the injection or emptying of liquid or fluid.

Making a judicious analysis of the above mentioned problems, we can conclude the insufflator equipment, as it is presented in the state of technique, presents an opportunity to its evolution aiming to conclusively solve its not so good aspects of constructive and operational optics.

### SUMMARY OF THE INVENTION

According to the described in Technical Background the petitioner idealized a new construction disposition introduced in insufflator equipment, which will be named in this document as “indeflator equipment”, where a new system to control the inflation or emptying of the balloon catheter was idealized, which eliminates the negative aspects identified in Technical Background, which:
It presents a simplified construction concept;
Easy assembly;
Significant durability;
Low index of functional non-conformity;
Easy handling; and
Efficient control of injection or emptying of liquid or fluid.

To make possible these features in its construction concept, particularly the control system to inflate or to empty the balloon catheter, it is basically composed by two gears, as a crog rail element (locking) and pinion element, manually set in motion by a trigger element, where the crog rail has the function of providing interference in the thread area of the embolus, preventing its free movement, when it is pulled or pushed. However the rotary movement is possible; thus, when the system is locked the embolus can advance and return if it is set in motion.

Thus the indeflator now claimed is used to inflate the balloon catheter only with the system locked and through a rotary movement of the embolus, since at the operational pressure of 450 psi a 100-kgf force acts to push the embolus to exit the syringe, and the user can not inflate the balloon through a simple action of directly push the embolus.

At its turn the indeflator is only handled if the system is unlocked, it is to say, with its trigger pulled, in a particular condition the user must aspirate contrast inside the syringe component, purge the air, or also to get a vacuum inside the balloon after the procedure.

### BRIEF DESCRIPTION OF THE DRAWINGS

In order to complement the current description and to get a better understanding of the characteristics of the claim of utility model, a set of figures is attached, where in an exemplified, but not limitative, way the preferred embodiment is represented to the indeflator equipment with a new control system to inflate or to empty the balloon catheter, where:

FIG. 1 is a perspective view of the indeflator equipment now claimed;

FIG. 2 is a general view of the indeflator equipment now claimed;

FIG. 3 is a detailed representation of the locking/unlocking system to inflate or to empty the balloon catheter now claimed, with the embolus locked;

FIG. 4 is a detailed representation of the locking/unlocking system to inflate or to empty the balloon catheter now claimed, with the embolus unlocked; and

FIG. 5 is a perspective view of the crog rail component.

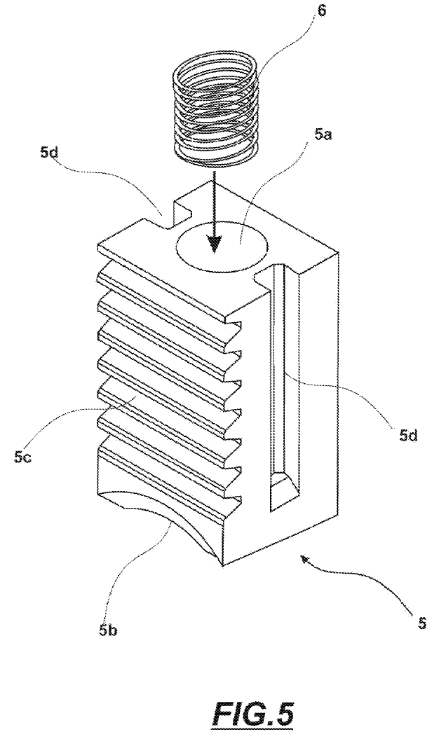

### DETAILED DESCRIPTION OF THE INVENTION

The following detailed description must be read and interpreted with the attached figures, highly diagrammatic, which represent one preferred realization form, not limiting the invention scope, which is only limited by the claims.

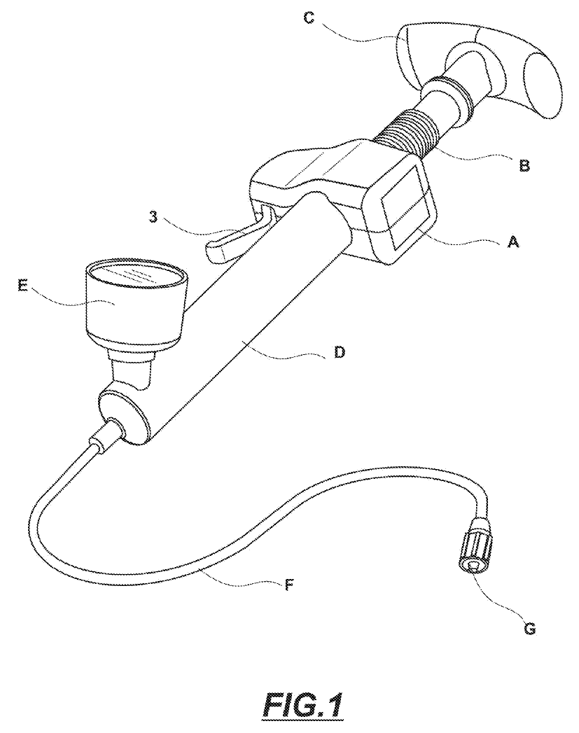

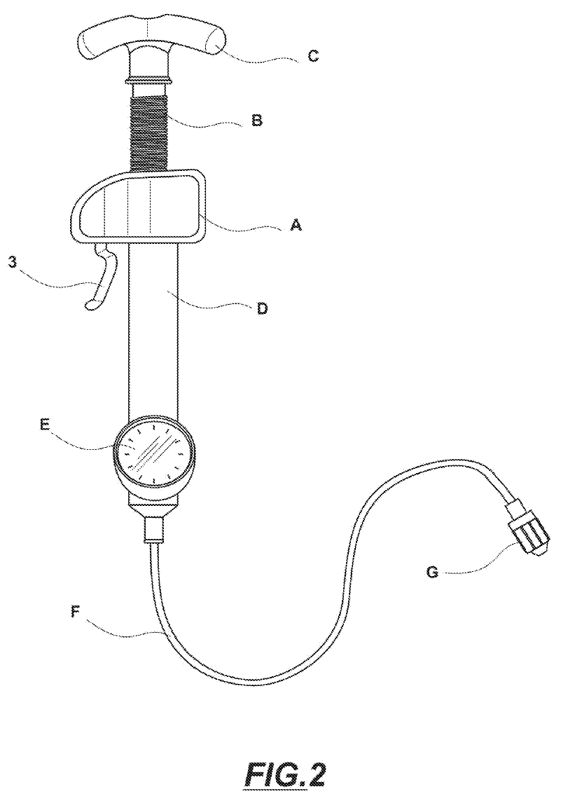

Related to the figures the current demand of patent of utility model is related to a constructive disposition introduced in indeflator equipment applied in coronary angioplasty intervention, whose construction concept is presented in FIGS. 1 and 2, being composed of a locking/unlocking system (A), installed in a way to interfere in the thread region of the embolus component (B), which presents in its end a hold element (C), whose anatomical design makes comfortable the use by the professional who must “push” the embolus (B) when inflating the balloon catheter is necessary.

Additionally the syringe frame (D) starts from the adjacent wall of the framework (1) of the locking/unlocking system (A), in whose frontal part is installed a manometer component (E) with the function of providing pressure control during the liquid or fluid administration.

Also in the frontal portion of the syringe frame (D) a connection of a flexible pipe (F) is made to provide the liquid or fluid flow, both to inflate and to empty the balloon catheter.

At its turn, at the end of the flexible pipe (F) there is a luer-type clamp element (G), which must be connected to the balloon catheter.

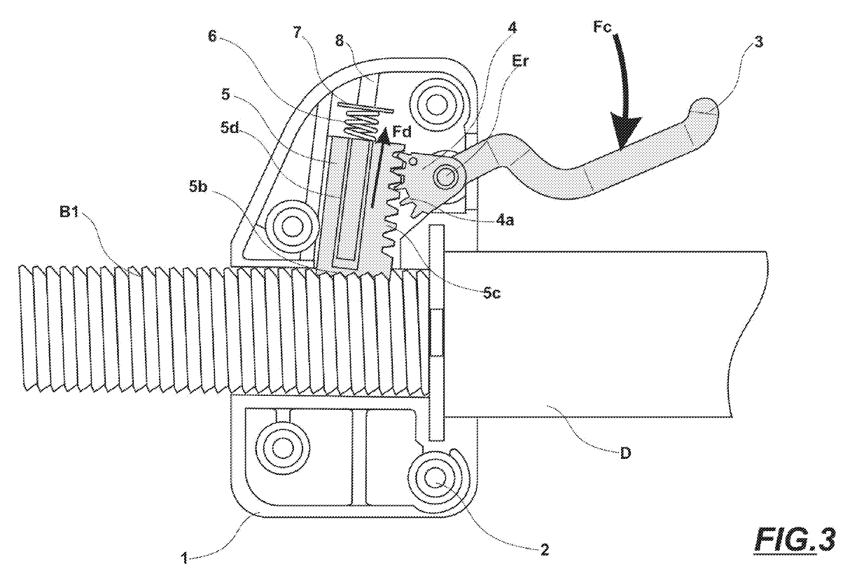

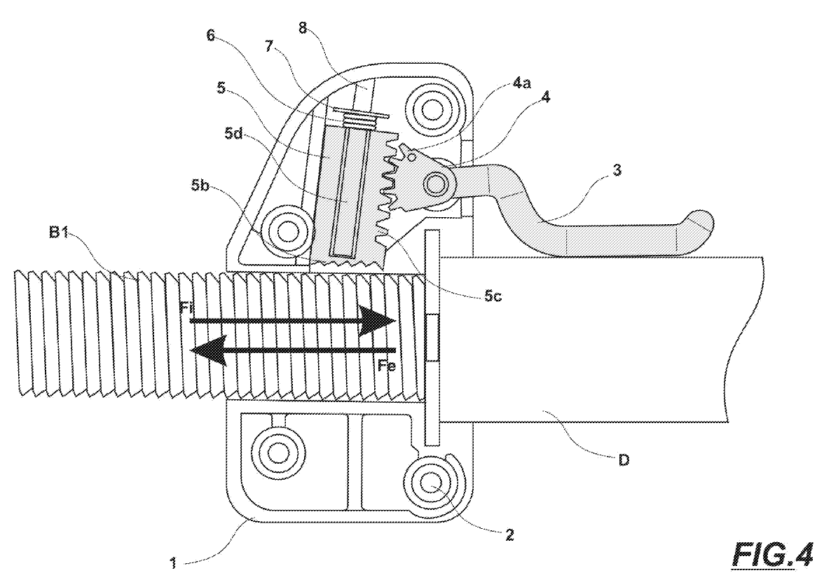

The locking/unlocking system (A) presents a construction concept, illustrated in FIGS. 3 and 4, composed by a framework (1), with two parts, which are closed by fixation elements disposed at the towers set (2), having such framework the function of accommodate the mechanism of the mentioned locking/unlocking system.

The mechanism of the locking/unlocking system (A) is composed by a trigger component (3), designed with anatomical project, which presents one free end, operated by the professional, with the opposite end extended to the interior of the framework (1). At this end the pinion component (4) is assembled, which is formed by a triangular frame with rounded edges, being the connection to the trigger component (3) made by one of these edges, and with the opposite lateral (4a) to this fixing edge presenting a circumference arc profile provided with a multiplicity of teeth, thus configuring a gear.

To interfere with the teeth of the opposite lateral (4a) of the pinion component (4) the crog rail component (5) is assembled, with a rectangular shape, such interference region having one lateral side (5c), with rectilinear profile, defining in all length a multiplicity of teeth, which configures one linear gear, designed according to the teeth of the opposite lateral (4a) of the pinion component (4).

The crog rail component (5) also presents a channel (5d), in each one of its lateral faces, with rectilinear profile of square section, which has the function of receiving the guide axis (8), also with square profile and fixed to the lateral internal wall of each one of the pieces that form the framework (1). Also related to the crog rail (5), it has in its inferior part (5b), a multiplicity of teeth, whose function is to provide interference with the external thread region (B1) of the embolus (B).

The superior part of the crog rail (5) presents a channel (5a) in which one is accommodated a helicoidal spring (6), having its superior part limited by a baffle plate (7) in a rectilinear plate shape, being part of the framework (1).

Its functional concept can be better understood with FIGS. 3 and 4, where in FIG. 3 the locking/unlocking system (A) is represented in the locked position. In this condition it is possible to verify the trigger component (3) with free end lifted, being the multiplicity of teeth of the inferior part (5b) of the crog rail (5), this one in interference condition at the external thread region (B1) of the embolus (B), where is possible to verify that the helicoidal spring component (6) is not tensioned, it is to say, it is totally extended.

When we desire to inflate or to empty the balloon catheter, we apply a compression force (Fc)—see FIG. 4—at the free end of the trigger (3), conferring to the pinion component (4) a rotary movement around its rotary axis (Er).

This movement makes the multiplicity of teeth of the opposite lateral (4a) or gear interferes directly in the multiplicity of teeth of the lateral wall (5c) of the crog rail (5), making a displacement force (Fd) provokes the displacement to the top of such crog rail (5), movement directed by the pair of guide axis elements (8).

This new crog rail position (5) avoid interference of the multiplicity of teeth defined in the inferior part (5b) at the thread region (B1) of the embolus (B), allowing it receives a force to inflate (Fi) or a pressure to empty (Fe) the balloon catheter.

For all described and illustrated we can see the indeflator equipment with a new locking/unlocking system accomplishes the requirements of utility model according to the Industrial Property Law, deserving as consequence the respective privilege.

1. Constructive disposition introduced in indeflator equipment applied in coronary angioplasty intervention characterized by an embolus component (B) with external thread region (B1), whose end presents a hold element (C), which is assembled in the internal diameter of the syringe frame (D), in whose frontal part a manometer component (E) and a flexible pipe (F) are assembled, and the free end of the flexible pipe receives a clamp (G), and in the intermediate part between the embolus (B) and the syringe frame (D) a locking/unlocking system (A) is assembled.

2. Constructive disposition introduced in indeflator equipment applied in coronary angioplasty intervention according to claim 1 characterized by the hold element (C) presenting an anatomical shape, which provides a more ergonomic condition.

3. Constructive disposition introduced in indeflator equipment applied in coronary angioplasty intervention according to claim 1 characterized by the locking/unlocking system (A) conferring a better control during the procedures to inflate or to empty the balloon catheter with specific liquid or fluid to coronary angioplasty intervention.

4. Constructive disposition introduced in indeflator equipment applied in coronary angioplasty intervention according to claim 3 characterized by the locking/unlocking system (A) presenting construction concept composed by a framework (1), formed with two pieces, which are closed with fixation elements at the towers set (2), whose interior receives the system mechanism, this one composed by a trigger component (3), with anatomical shape, which presents a free end, being that in the opposite end, and internal to the framework (1), one of the rounded edges of the pinion component (4) is fixed, in a triangular frame, whose opposite lateral (4a) to this edge has a circumference arc profile, with a multiplicity of teeth that are incased to the multiplicity of teeth of the lateral wall (5c) of the crog rail component (5), in a rectangular shape, whose inferior part (5b) is also formed by a multiplicity of teeth, and its central portion has a channel (5a), of rectilinear profile and cylindrical section, in which a helicoidal spring component (6) is encased, having in its lateral faces a pair of channels (5d) of rectilinear profile, in which the pair guide axis (8), of cylindrical profile, this one fixed to the lateral internal framework wall (1), of each one of its parts, having a helicoidal spring component (6) limited in its inferior portion by the superior part of the crog rail (5) and in its superior portion by a baffle plate (7).

5. Constructive disposition introduced in indeflator equipment applied in coronary angioplasty intervention according to claim 4 where the operation to inflate or to empty the balloon catheter with liquid or fluid is made impossible due to the non-operation of the embolus (B) over the syringe (D), independently of applying a force at the hold element (C) to push or to pull it, respectively, being in such condition the locking/unlocking system (A) is in the locking position characterized by presenting the trigger (3) with the free end lifted, with the teeth of the opposite lateral (4a) of the pinion (4) interfering with the teeth of the lateral wall (5c) of the crog rail (5), this one fixed in a position that keeps the multiplicity of teeth of its inferior part (5b) directly interfering over the external thread region (B1) of the embolus (B), with the helicoidal spring (6) in the extended position.

6. Constructive disposition introduced in indeflator equipment applied in coronary angioplasty intervention according to claim 4 where the operation to inflate or to empty the balloon catheter with liquid or fluid is made possible by the movement of the embolus (B) over the syringe (D), by applying a force to inflate (Fi) or to empty (Fe) in the hold element (C) through pushing or pulling it, respectively, being to such condition the locking/unlocking system (A) remains in unlocking position characterized by presenting the trigger (3) with the free end lowered, adjacent to the syringe frame (D), where the pinion (4) presents its opposite lateral (4a) with a multiplicity of teeth interfering with the teeth of the lateral wall (5c) of the crog rail (5), being this one remains in position dislocated to the top, with the helicoidal spring (6) in tensioned position and the external thread region (B1) of the embolus (B) free.
